(19) United States
(12) Patent Application Publication
(10) Pub. No.: US 2007/0093492 A1
(43) Pub. Date: Apr. 26, 2007

(54) Pyrrolidine derivatives

(76) Inventors: Weir-Torn Jiaang, Taichung (TW); Yu-Sheng Chao, Warren, NJ (US); Ting-Yueh Tsai, Tainan City (TW)

Correspondence Address: FISH & RICHARDSON PC, P.O. BOX 1022, MINNEAPOLIS, MN, 55440-1022, US

(21) Appl. No.: 11/545,070

(22) Filed: Oct. 6, 2006

Related U.S. Application Data
(63) Continuation-in-part of application No. 11/077,551, filed on Mar. 9, 2005.
(60) Provisional application No. 60/551,419, filed on Mar. 9, 2004.
(60) Provisional application No. 60/617,684, filed on Oct. 12, 2004.

(51) Int. Cl. A61K 31/498; A61K 31/4745; A61K 31/4709; A61K 31/427; A61K 31/4035; A61K 31/401; C07D 487/04; C07D 498/02; C07D 403/02

(52) U.S. Cl. 514/249; 514/365; 514/310; 514/414; 544/350; 548/200; 546/146; 546/114; 548/465; 514/301; 514/423; 548/530

(57) ABSTRACT

Pyrrolidine compounds described herein and methods for using them to inhibit dipeptidyl peptidase IV or treat Type II diabetes.

### CROSS REFERENCE TO RELATED APPLICATIONS

This application is a continuation-in-part of U.S. application Ser. No. 11/077,551, filed Mar. 9, 2005, which claims priority to U.S. Provisional Application Ser. No. 60/551,419, filed Mar. 9, 2004, and U.S. Provisional Application Ser. No. 60/617,684, filed Oct. 12, 2004. The contents of all of the three prior applications are hereby incorporated by reference in their entireties.

### BACKGROUND

Glucagon-like peptide-1 (GLP-1) is a gut hormone produced by intestinal endocrine L-cells in response to nutrient ingestion. GLP-1 inhibits glucagon secretion and stimulates glucose-dependent insulin release from the pancreas. It was observed that administration of GLP-1 significantly lowered blood glucose levels in Type II diabetes patients (Zander M, et al. Lancet 2002, 359: 824-830).

However, GLP-1, whether endogenously or exogenously administered, degrades rapidly. (Kieffer T. J., et al. Endocrinology 1995, 136: 3585-3596; and Mentlein R, et al. Eur. J. Biochem. 1993, 214: 829-839). The degradation is attributable to dipeptidyl peptidase IV (DPP-IV), a member of the prolyl peptidase family. Recent clinical data indicate that inhibiting DPP-IV resulte in enhanced insulin secretion, reduced plasma glucose concentrations, and improved pancreatic β-cell function (Pederson R. A., et al. Diabetes 1998, 47: 1253-1258; and Ahren B, et al. Diabetes Care 2002, 25: 869-875). Thus, inhibitors of DPP-IV are potential drug candidates for Type II diabetes.

### SUMMARY

This invention is based on a surprising discovery that a group of pyrrolidine compounds inhibit DPP-IV.

One aspect of this invention relates to pyrrolidine compounds of the following general formula: 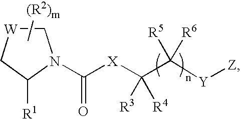
wherein R¹ is H or CN; R² is H, halo, nitro, cyano, amino, hydroxy, alkyl, haloalkyl, alkoxy, aryloxy, aralkyl, cyclyl, heterocyclyl, aryl, or heteroaryl;-each of R³, R⁴, R⁵, and R⁶, independently, is H, halo, nitro, cyano, amino, hydroxy, alkyl, haloalkyl, alkoxy, aryloxy, aralkyl, cyclyl, heterocyclyl, aryl, or heteroaryl; or R³ and R⁴, together with the carbon atom to which they are attached, or R⁵ and R⁶, together with the carbon atom to which they are attached, are a 3-8 membered ring, optionally having 1 or 2 heteroatoms and optionally substituted with halo, CN, NO₂, —OR^{a}, alkyl, aryl, heteroaryl, haloalkyl, —OR^{a}, —C(O)R^{a}, —SR^{a}, —S(O)R^{a}, —S(O)₂R^{a}, —NR^{a}R^{a′}, —C(O)OR^{a}, —C(O)NR^{a}R^{a′}, —OC(O)R^{a}, —NR^{a}C(O)R^{a′}, —NR^{a}C(O)OR^{a′}, or —NR^{a}C(O)NR^{a′}R^{a″}, or optionally fused with one of cyclyl, heterocyclyl, aryl, and heteroaryl, each of R^{a}, R^{a′}, and R^{a″}, independently, being H, alkyl, or aryl; m is 0, 1, 2, 3, 4, or 5; n is 0, 1, or 2; W is CR^{b}R^{b′}, NR^{b}, O, or S, in which each of R^{b} and R^{b′}, independently, is H, halogen, alkyl, or aryl; X is O, S, or CR^{c}(NR^{c′}R^{c″}), in which each of R^{c}, R^{c′}, and R^{c″}, independently, is H, alkyl, or aryl; Y is 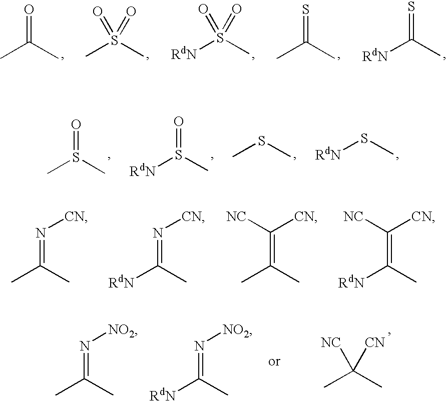
in which R^{d} is H, alkyl, or aryl; and Z is NR^{e}R^{e′}, in which each of R^{e} and R^{e′}, independently, is H, alkyl, alkoxyalkyl, haloalkyl, cyclyl, heterocyclyl, aryl, aralkyl, heteroaryl, or heteroaralkyl; or NR^{e}R^{e′}, together, is a 3-8 membered ring having 1 or 2 heteroatoms, optionally substituted with halo, CN, NO₂, —OR′, alkyl, aryl, heteroaryl, haloalkyl, —OR′, —C(O)R′, —SR′, —S(O)R′, —S(O)₂R′, —NR′R″, —C(O)OR′, —C(O)NR′″, —OC(O)R′, —NR′C(O)R″, —NR′C(O)OR″, or —NR′C(O)NR″R′R″, or optionally fused with one of cyclyl, heterocyclyl, aryl, and heteroaryl, each of R′, R″, and R′″, independently, being H, alkyl, or aryl.

Referring to the just-described pyrrolidine compounds, a subset features that X is CHNH₂ and each of R³, R⁴, R⁵, and R⁶, independently, is H, halo, nitro, cyano, amino, hydroxy, alkyl, haloalkyl, alkoxy, aryloxy, aralkyl, cyclyl, heterocyclyl, aryl, or heteroaryl. In these compounds, R³ and R⁴ can be CH₃ and each of R⁵ and R⁶ can be H; or each of R³, R⁴, R⁵, and R⁶ can be H; or R³ can be CH₃ and each of R⁴, R⁵, and R⁶ can be H. Another subset of the pyrrolidine compounds features R³ and R⁴ together with the carbon atom to which they attached are a cyclopropyl ring. A further subset features that Y is C(O).

Another aspect of this invention relates to pyrrolidine compounds of 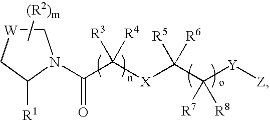
wherein R¹ is H or CN; each of R², R³, R⁴, R⁵, R⁶, R⁷, and R⁸, independently, is H, halo, nitro, cyano, amino, hydroxy, alkyl, haloalkyl, alkoxy, aryloxy, aralkyl, cyclyl, heterocyclyl, aryl, or heteroaryl; m is 0, 1, 2, 3, 4, or 5; n is 0, 1, 2, 3, or 4; o is 0, 1, 2, or 3; W is CR^{a}R^{a′}, NR^{a}, O, or S, in which each of R^{a} and R^{a′}, independently, is H, halogen, alkyl, or aryl; X is NR^{b}, in which R^{b} is H, alkyl, or aryl; Y is 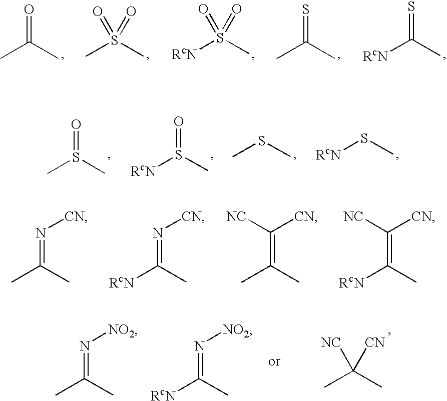
in which R^{c} is H, alkyl, or aryl; and Z is NR^{d}R^{d′}, in which R^{d} is a 3-8 membered monocyclic ring optionally substituted with halo, CN, NO₂, —OR′, alkyl, aryl, heteroaryl, haloalkyl, —OR′, —C(O)R′, —SR′, —S(O)R′, —S(O)₂R′, —NR′R″, —C(O)OR′, —C(O)NR′R″, —OC(O)R′, —NR′C(O)R″, —NR′C(O)OR″, or —R′C(O)NR″R^{40 ″; and R}^{d′} is H, alkyl, alkoxyalkyl, haloalkyl, aralkyl, or heteroaralkyl; each of R′, R″, and R′″, independently, being H, alkyl, or aryl.

Referring to the just-described pyrrolidine compounds, a subset features that n is 1 and o is 1; X is NH; W is CH₂ or CHF; R^{d} is a cyclopropyl ring substituted with an aryl or heteroaryl group; each of R³, R⁴, R⁷, and R⁸ is H; and each of R⁵ and R⁶ is CH₃. Another subset features that Y is C(O).

Shown below are exemplary compounds of this invention:

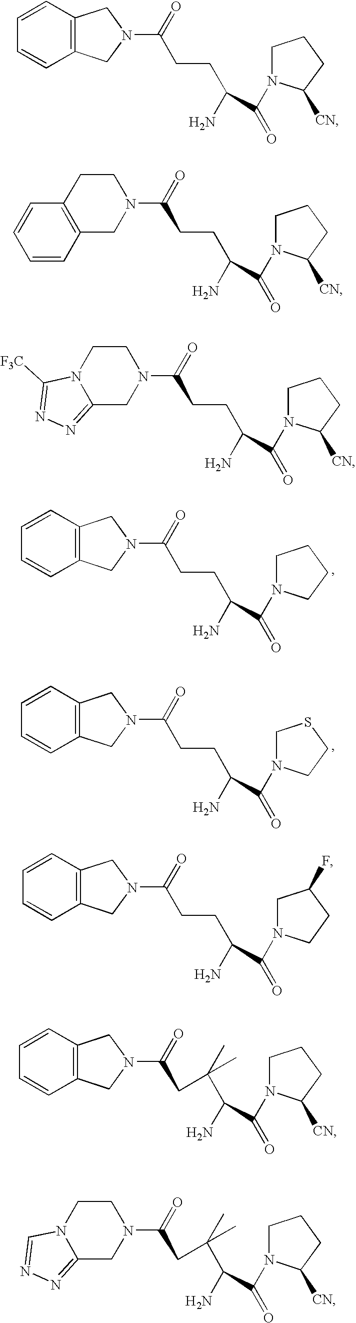

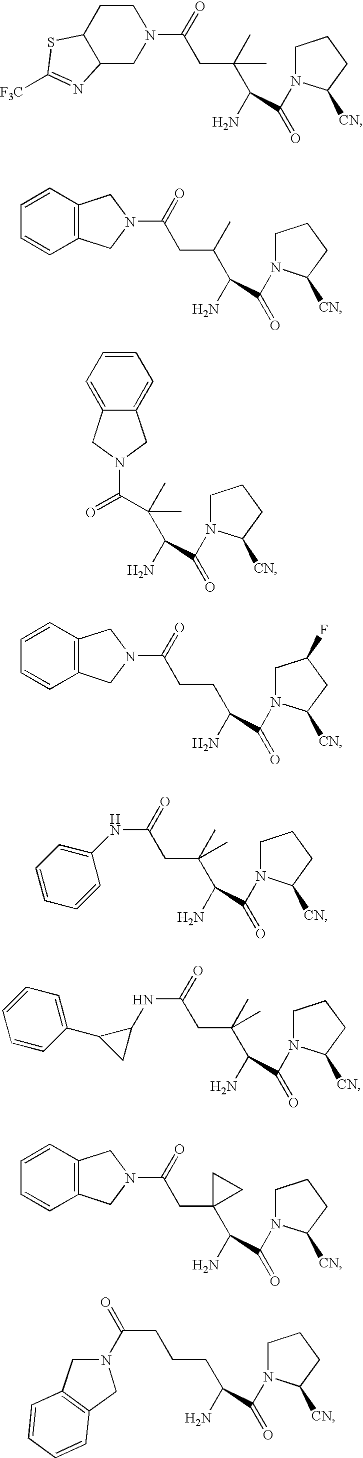

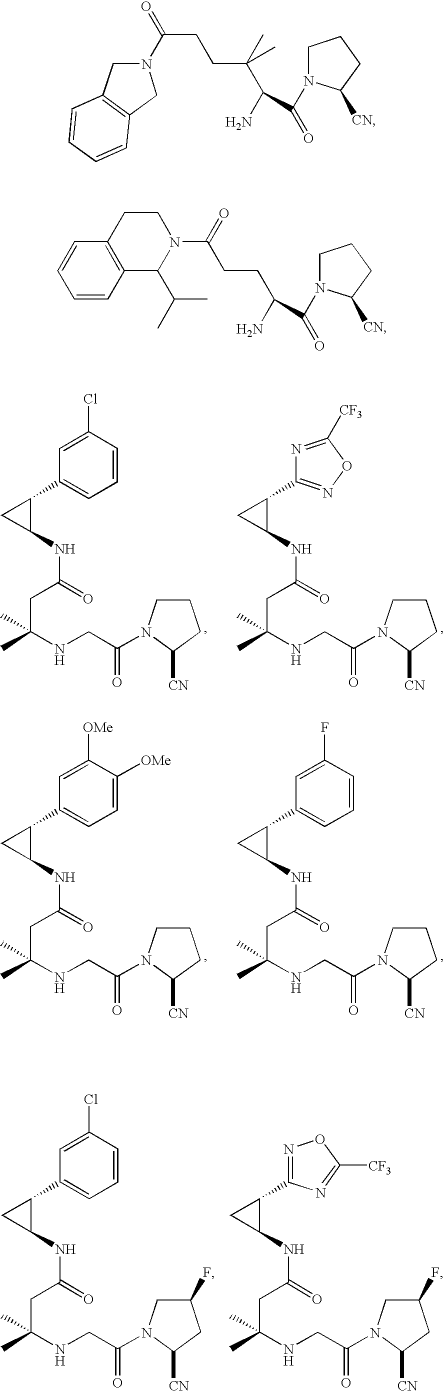

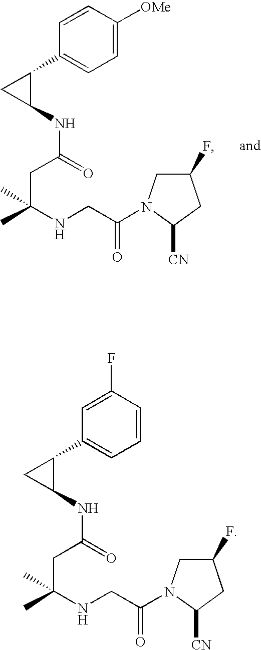

The term “alkyl” herein refers to a straight or branched hydrocarbon, containing 1-10 carbon atoms. Examples of alkyl groups include, but are not limited to, methyl, ethyl, n-propyl, i-propyl, n-butyl, i-butyl, and t-butyl. The term “alkoxy” refers to an —O-alkyl. The term “alkoxyalkyl” refers to an alkyl group substituted with one or more alkoxy groups. The term “haloalkyl” refers to an alkyl group substituted with one or more halo groups. The term “hydroxyalkyl” refers to an alkyl group substituted with one or more hydroxy groups.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl. The term “aryloxy” refers to an —O-aryl. The term “aralkyl” refers to an alkyl group substituted with an aryl group.

The term “cyclyl” refers to a saturated and partially unsaturated cyclic hydrocarbon group having 3 to 12 carbons. Examples of cyclyl groups include, but are not limited to, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl. The term “heteroaralkyl” refers to an alkyl group substituted with a heteroaryl group.

The term “heterocyclyl” refers to a nonaromatic 5-8 membered monocyclic, 8-12 membered bicyclic, or 11-14 membered tricyclic ring system having one or more heteroatoms (such as O, N, or S). Examples of heterocyclyl groups include, but are not limited to, piperazinyl, pyrrolidinyl, dioxanyl, morpholinyl, and tetrahydrofuranyl.

Alkyl, cyclyl, heterocyclyl, aryl, heteroaryl, aralkyl, heteroaralkyl, alkoxy, and aryloxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, heterocyclyl, in which alkyl, alkenyl, alkynyl, alkyloxy, aryl, heteroaryl cyclyl, and heterocyclyl may further substituted.

The monocyclic ring mentioned herein is either substituted or unsubstituted, but cannot be fused with another aromatic or non-aromatic ring.

The pyrrolidine compounds described above include their pharmaceutically acceptable salts and prodrugs, if applicable. Such a salt can be formed between a positively charged ionic group in an pyrrolidine compound (e.g., ammonium) and a negatively charged counterion (e.g., trifluoroacetate). Likewise, a negatively charged ionic group in a pyrrolidine compound (e.g., carboxylate) can also form a salt with a positively charged counterion (e.g., sodium, potassium, calcium, or magnesium). The pyrrolidine compounds may contain a non-aromatic double bond and one or more asymmetric centers. Thus, they can occur as racemic mixtures, single enantiomers, individual diastereomers, diastereomeric mixtures, and cis- or trans-isomeric forms. All such isomeric forms are contemplated.

The pyrrolidine compounds described above can be used to inhibit DPP-IV. Accordingly, another aspect of this invention relates to a method of inhibiting DPP-IV with one or more of the pyrrolidine compounds. As inhibition of DPP-IV results in reduced blood glucose levels and enhanced insulin secretion, the compounds of this invention can be also used to treat Type II diabetes. Thus, this invention further covers a method of treating Type II diabetes by administering to a subject in need thereof an effective amount of one or more of the pyrrolidine compounds.

Also within the scope of this invention is a pharmaceutical composition containing one or more of the above-described pyrrolidine compounds and a pharmaceutically acceptable carrier, as well as use of the composition for the manufacture of a medicament for treating Type II diabetes.

The details of many embodiments of the invention are set forth in the description below. Other features, objects, and advantages of the invention will be apparent from the description and the claims.

### DETAILED DESCRIPTION

The pyrrolidine compounds of this invention can be synthesized by methods well known in the art. Six exemplary synthetic routes are shown in Schemes 1-6 below.

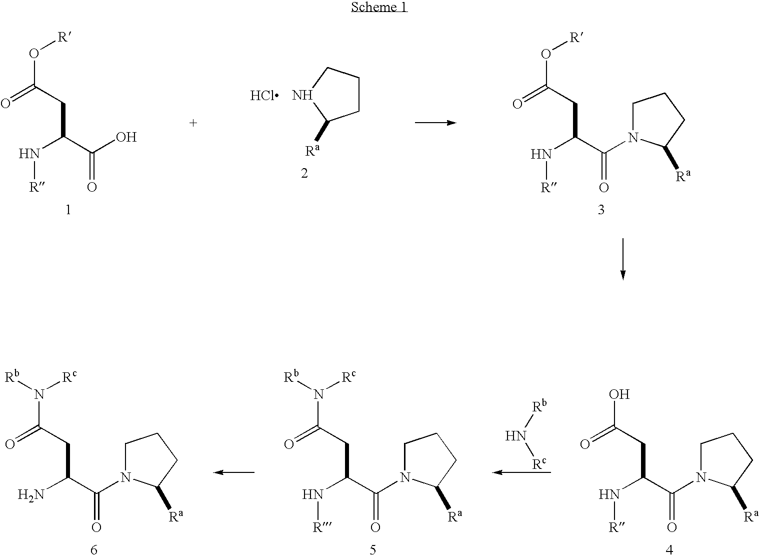

In Scheme 1, the starting compound is amino-substituted dicarboxylic acid (1) in which an amino group and one of two carboxy groups are protected. This compound is reacted with 2-substituted pyrrolidine hydorchloride salt (2) to give monoamide intermediate (3). Note that synthesis of 2-substituted pyrrolidine hydrochloride salt (2) is well known in the art. For example, pyrrolidine-2-carbonitrile hydrochloride salt can be prepared by the procedure described in Bioorg. Med. Chem. Lett. 1996, 6: 1163. Removing the carboxy protected group of the intermediate (3) affords monoamide monoacid compound (4), which subsequently is coupled with amine to provide diamide compound (5). Deprotection of compound (5) provides desired pyrrolidine compound (6).

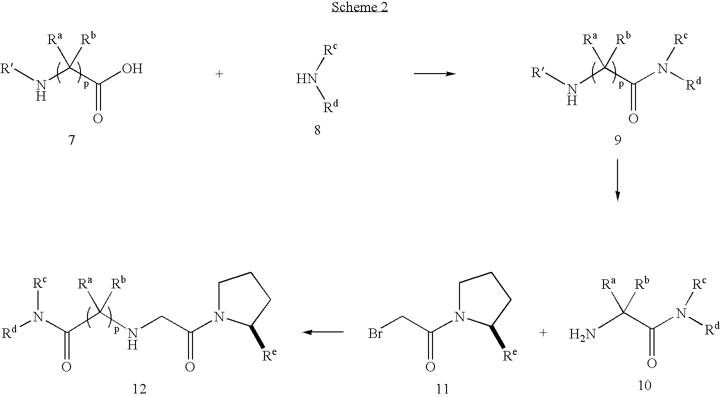

Scheme 2 illustrates another synthetic route for synthesizing pyrrolidine compounds. The starting compound is α-amino acid (7), in which the amino group is protected. This compound is coupled with amine (8) to give amide compound (9). Compound (9) is deprotected and subsequently reacted with 1-(2-bromo-acetyl)pyrrolidine (11) to afford desired pyrrolidine compound (12). Note that 1-(2-bromo-acetyl)pyrrolidine (11) can be prepared by methods well known in the art. See, e.g., J. Med. Chem. 2003, 46: 2774.

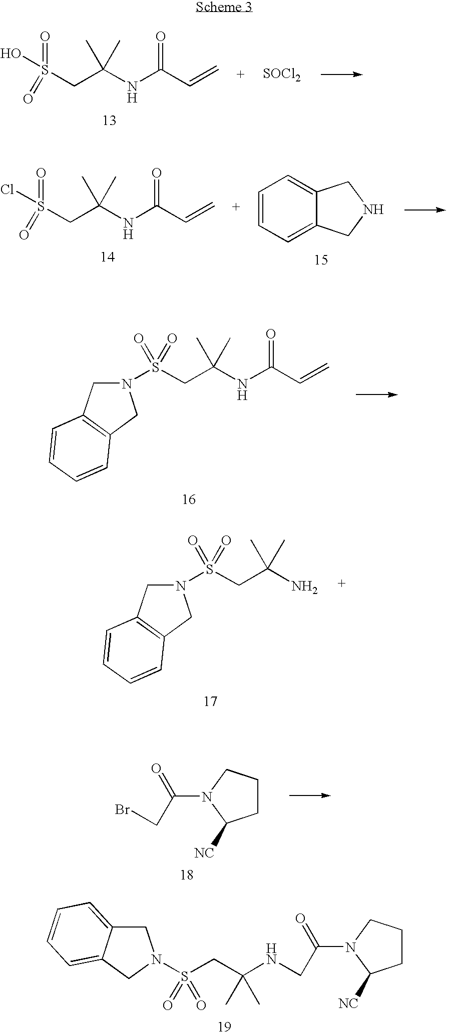

In Scheme 3, the starting compound is N-protected 2-amino-2-methyl-propane-sulfanoic acid (13), which is commuercially available. It is reacted with sulfuryl chloride and then with 2,3-dihydroisoindole to give sulfonyl amide (16), which is subsequently deprotected to afford amino compound (17). This amino compound is coupled with β-bromo amide (18) to form desired pyrrolidine compound (19).

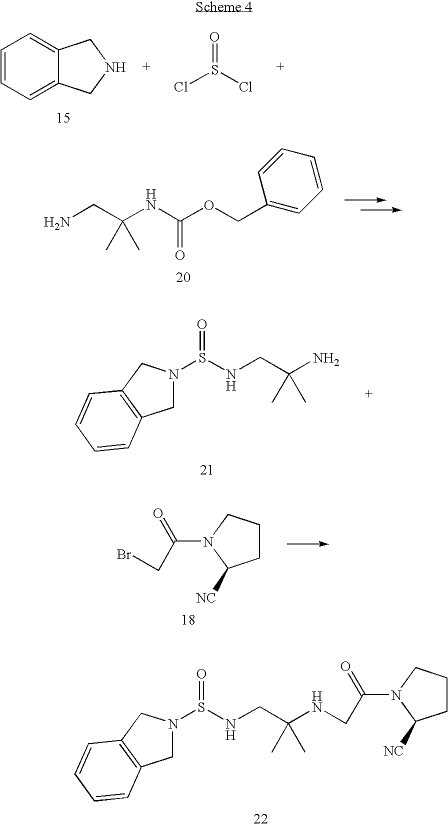

In Scheme 4, thionyl chloride is reacted with 2,3-dihydroisoindole (15) and (2-amino-1,1-dimethyl-ethyl)-carbamic acid benzyl ester (20), sequentially. The product (not shown), a protected amino compound, is deprotected to afford free amino compound (21), which is subsequently coupled with β-bromo amide (18) to form desired pyrrolidine compound (22).

Similarly, two additional pyrrolidine compounds of this invention, i.e., compounds (26) and (29), can be prepared following analoguous procedures as shown in Schemes 5 and 6 below. Starting material (24) is reportedly synthesized before. See, e.g., Boehringer M. et al., WO 2003037327.

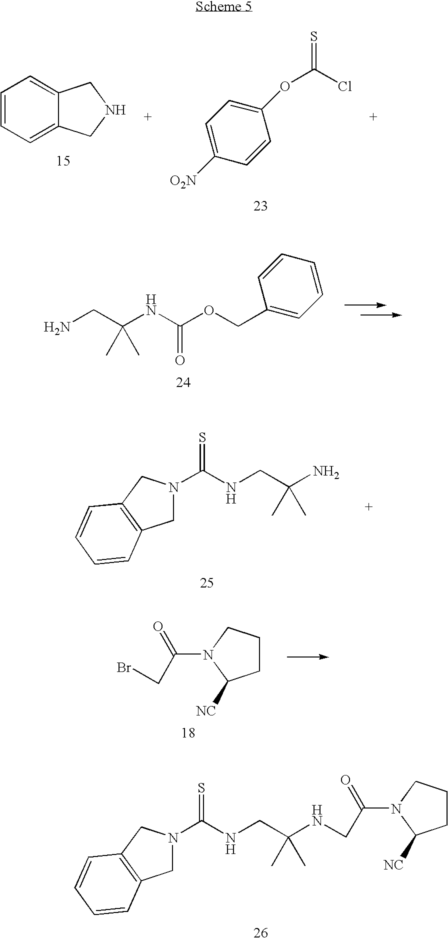

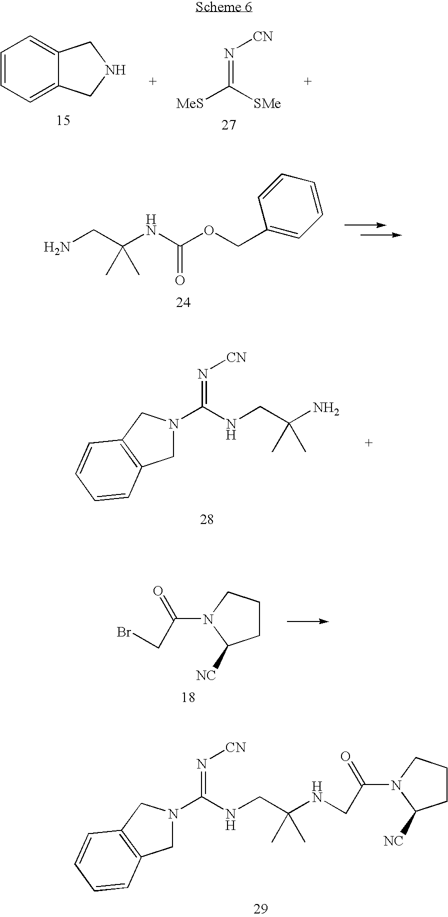

Scheme 7 below illustrates synthesis of a cyclopropyl-containing pyrrolidine compound. Starting material (30) is a N-protected β-amino acid. It reacts with cyclopropyl amine in the presence of a coupling agent (e.g., dicyclohexylcarbodiimide), followed by deprotection, to provide N-cyclopropyl amide (31), which has a free amino group. The amide is then coupled with pyrrolidine (32) to form cyclopropyl-containing pyrrolidine (33). N-protected β-amino acid (30) and pyrrolidine (32) can be prepared by known methods. See, e.g., J. Med. Chem. 2006, 49, 373; J. Med. Chem. 1988, 31, 92; and J. Med. Chem. 2002, 45, 2362.

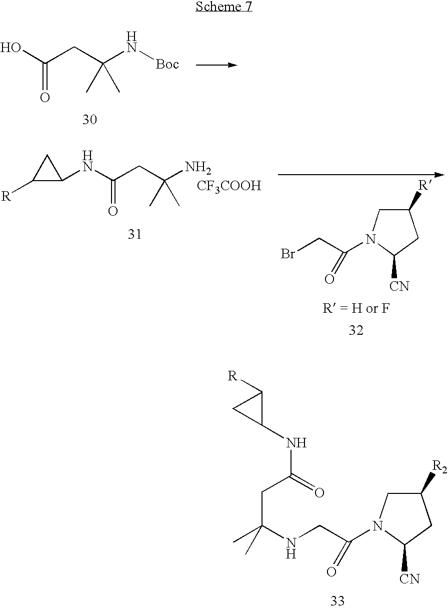

Scheme 8 below shows synthesis of a pyrrolidine compound having a longer chain (i.e., 3 carbon atoms between carbonyl groups). Also this chain can be either substituted or unsubstituted.

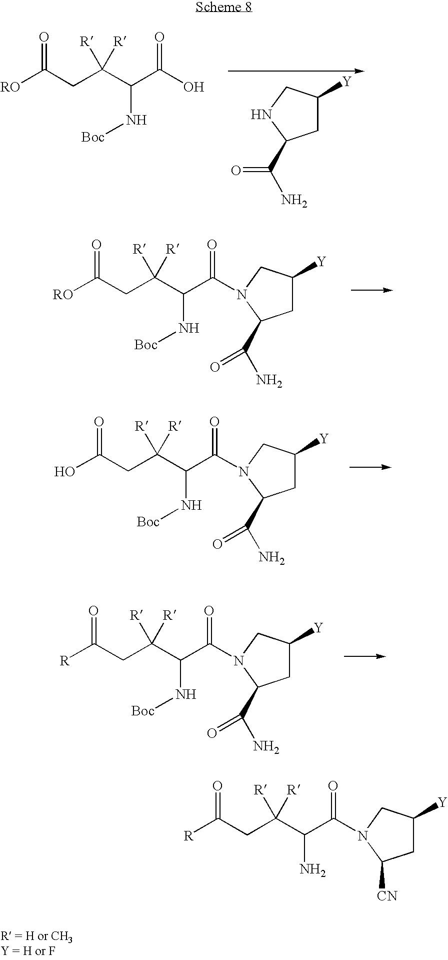

The above eight schemes are provided only for illustrative purposes. A skilled person in the art, in view of them, would be able to synthesize all the pyrrolidine compounds of this invention with any necessary modifications within his or her skill. Synthetic chemistry transformations and protecting group methodologies (protection and deprotection) useful in synthesizing applicable pyrrolidine compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Pyrrolidine compounds thus obtained can be further purified by column chromatography, high performance liquid chromatography, or crystallization.

This invention covers a method for inhibiting DPP-IV by contacting it with an effective amount of one or more of the pyrrolidine compounds described above. This invention also covers a method for treating Type II diabetes by administering to a subject in need thereof an effective amount of one or more of the pyrrolidine compounds described above. The term “treating” refers to application or administration of the pyrrolidine compound to a subject, who has Type II diabetes, a symptom of Type II diabetes, or a predisposition toward Type II diabetes, with the purpose to cure, heal, alleviate, relieve, alter, remedy, ameliorate, improve, or affect the disease, the symptom, or the predisposition. “An effective amount” refers to the amount of the pyrrolidine compound which is required to confer the desired effect on the subject. Effective amounts vary, as recognized by those skilled in the art, depending on route of administration, excipient usage, and the possibility of co-usage with other therapeutic treatments such as use of other active agents.

To practice the treatment method of the present invention, a composition having one or more of the pyrrolidine compounds describe above can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol and water. In addition, fixed oils are conventionally employed as a solvent or suspending medium (e.g., synthetic mono—or diglycerides). Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active pyrrolidine compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active pyrrolidine compound. Examples of other carriers include colloidal silicon oxide, magnesium stearate, cellulose, sodium lauryl sulfate, and D&C Yellow # 10.

The pyrrolidine compounds of this invention can be preliminarily screened by an in vitro assay for one or more of their desired activities, e.g., inhibiting DPP-IV. Compounds that demonstrate high activities in the preliminary screening can further be screened for their efficacy by in vivo assays. For example, a test compound can administered to an animal (e.g., a mouse model) having type II diabetes and its therapeutic effects are then accessed. Based on the results, an appropriate dosage range and administration route can also be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All of the publications, including patents, cited herein are hereby incorporated by reference in their entirety.

### EXAMPLE 1

### Preparation of 1-[2-amino-5-(1,3-dihydro-isoindol-2-yl)-5-oxo-pentanoyl]-4-fluoro-pyrrolidine-2-carbonitrile trifluoroacetic acid

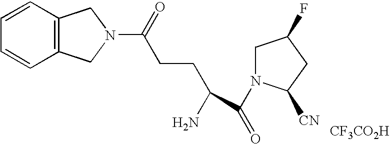

A solution of butoxycarbonylamino-L-glutamic acid 5-methyl ester (0.522 g, 2 mmol) and N-hydroxysuccinimide (0.23 g, 2 mmol) in 6 ml dichloromethane (DCM)/1,4-dioxane (2:1) was cooled in an ice-water bath. To this was added N,N′-dicyclohexylcarbodiimide (DCC, 0.45 g, 2.2 mmol). The mixture was stirred at room temperature for 1 hour, and then 4-fluoro-pyrrolidine-2-carboxylic acid amide (0.264 g, 2 mmol) was added. After stirred for 4 hours at room temperature, the mixture was filtered to remove DCC, and then washed with DCM. The combined organic solution was washed with 10% aqueous citric acid solution and saturated aqueous NaHCO₃ solution, dried over MgSO₄, and concentrated in vacuo. Purification by flash column chromatography (eluted with DCM/MeOH=98/2 to 95/5) afforded 4-tert-butoxycarbonylamino-5-(2-carbamoyl-4-fluoro-pyrrolidin-1-yl)-5-oxo-pentanoic acid methyl ester (85%) as a foam.

A solution of 4-tert-butoxycarbonylamino-5-(2-carbamoyl-4-fluoro-pyrrolidin-1-yl)-5-oxo-pentanoic acid methyl ester (0.361 g, 1 mmol) in THF/H₂O was cooled in an ice bath. To this was added LiOH (0.048 g, 2 mmol). After stirred at the low temperature for 3 hours, the reaction solution was partitioned with ethyl acetate and 10% aqueous citric acid. The organic layer was dried over MgSO₄ and concentrated in vacuo to give 4-tert-butoxycarbonylamino-5-(2-carbamoyl-4-fluoro-pyrrolidin-1-yl)-5-oxo-pentanoic acid without further purification.

A solution of the above-obtained compound and N-hydroxysuccinimide (0.361 g, 1 mmol) in 8 ml DCM/1,4-dioxane (2/1) was cooled in an ice-water bath. To this was added DCC (0.23 g, 1.1 mmol). After the mixture was stirred at room temperature for 1 hour, 2,3-dihydro-1H-isoindole (0.18 g, 1.5 mmol) was added. The reaction mixture was stirred at room temperature for 4 hours, filtered to remove DCC, and then washed by DCM. The combined organic solution was washed with 10% aqueous citric acid solution and saturated aqueous NaHCO₃ solution, dried over MgSO₄, and concentrated in vacuo. Purification by flash column chromatography (eluted with CH₂Cl₂/MeOH from 98/2 to 95/5) afforded [1-(2-Carbamoyl-4-fluoro-pyrrolidine-1-carbonyl)-4-(1,3-dihydro-isoindol-2-yl)-4-oxo-butyl]-carbamic acid tert-butyl ester (83%) as a foam.

The above-obtained compound (0.462 g, 1 mmol) and imidazole (0.102 g, 1.5 mmol) were dissolved in pyridine (4 ml). The solution was cooled to −20° C. Phopsphoryl chloride (0.23 ml, 2.5 mmol) was added dropwise over a period of 2 minutes and the resulting mixture was stirred at −20° C. for 1 hour. Pyridine was removed by a high vacuum pump, the crude product was washed with 10% aqueous citric acid solution and saturated aqueous NaHCO₃ solution, dried over MgSO₄, and concentrated in vacuo. Purification by flash column chromatography (eluted with hexane/EA=1/3) yielded N-t-BOC-[2-Amino-5-(1,3-dihydro-isoindol-2-yl)-5-oxo-pentanoyl]-4-fluoro-pyrrolidine-2-carbonitrile (93%) as a foam. This compound was ten dissolved in cool trifluoroacetic acid (2 ml) and stirred at room temperature for 10 minutes and concentrated in vacuo for over night. The title compound was obtained as a taffy.

¹H NMR (CDCl₃): 8.10-7.23 (m, 4H), 5.50 (s, 0.5 H), 5.34 (s, 0.5 H), 5.01 (d, J=9.3 Hz, 1H), 4.86-4.73 (m, 4H), 4.49 (brs, 1H), 4.07-3.80 (m, 2H), 2.78 (brs, 2H), 2.63 (t, J=15.6 Hz, 1H), 2.50-2.42 (m, 1H), 2.36-2.21 (m, 2H); MS (ESI) m/z: 345.1 (M+H)⁺, 367.1 (M+Na)⁺.

### EXAMPLE 2

### Preparation of 1-[2-amino-5-(1,3-dihydro-isoindol-2-yl)-5-oxo-pentanoyl]-pyrrolidine-2-carbonitrile trifluoroacetic acid

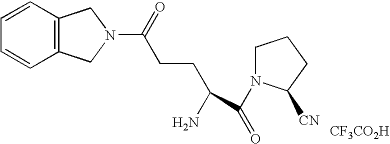

The title compound was prepared in a similar manner as described in Example 1.

¹H NMR (CD₃OD): 7.34-7.27 (m, 4H), 4.87-4.81 (m, overlapped singlet at 4.86, 5H), 4.40 (t, J=5.7 Hz, 1H), 3.87-3.79 (m, 1H), 3.73-3.65 (m, 1H), 2.77 (dd, J=7.2, 5.4 Hz, 2H), 2.37-2.12 (m, 6H); MS (ESI) m/z: 327.3 (M+H)⁺, 349.3 (M+Na)⁺.

### EXAMPLE 3

### Preparation of N-[2-(3-Chloro-phenyl)-cyclopropyl]-3-[2-(2-cyano-pyrrolidin-1-yl)-2-oxo-ethylamino]-3-methyl-butyramide

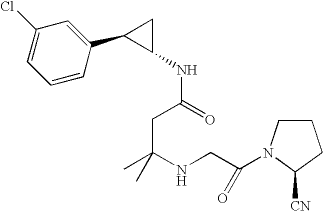

A solution of 3-tert-butoxycarbonylamino-3-methyl-butyric acid (2.17 g, 10 mmol) and N-hydroxysuccinimide (1.15 g, 10 mmol) in 20 mL DCM/1,4-dioxane (2:1) was cooled in an ice-water bath. To this was added DCC (2.3 g, 11 mmol). The mixture was stirred at room temperature for 1 hour, and then 2-(3-chloro-phenyl)-cyclopropylamine 2.5 g, 15 mmol) was added. After stirred for 4 hours at room temperature, the mixture was filtered to remove DCC, and then washed with DCM. The combined organic solution was washed with 10% aqueous citric acid solution and saturated aqueous NaHCO₃ solution, dried over MgSO₄, and concentrated in vacuo. Purification by flash column chromatography (eluted with Hexane/CH₂Cl₂/EA=4:5:1) yielded 3-N-t-butoxycarbonyl-amino-N′-((1R,2S)-2-(3-chlorophenyl)cyclopropyl)-3-methylbutanamide 2,2,2-trifluoroacetate (88%) as a foam. This compound was dissolved in cool trifluoroacetic acid (2 ml). The resulting solution was stirred at room temperature for 10 minutes and vacuumed overnight. 3-Amino-N-((1R,2S)-2-(3-chlorophenyl)cyclopropyl)-3-methylbutanamide was obtained as a taffy.

To a solution of the above-obtained compound (0.38 g, 1 mmol) in dry THF (6 ml) was added K₂CO₃ (1.38 g, 10 mmol), and the reaction was stirred at room temperature for 1.5 hours. The resulting mixture was filtered to remove K₂CO₃, and the filtrate was concentrated in vacuo. After the oily residue was diluted with THF (3 ml), (S)-1-(2-bromoacetyl)pyrrolidine-2-carbonitrile was added dropwise. The resultant mixture was stirred at room temperature overnight, washed with saturated aqueous NaHCO₃ solution, dried over MgSO₄, and concentrated in vacuo. Purification by flash column chromatography (eluted with CH₂Cl₂/MeOH: 96:4) yielded the title compound 3 as a light yellow oil.

¹H NMR (CDCl₃)(5/1 mixture of trans/cis amide isomers): 8.65 (d, J=3.3 Hz, 5/6H), 8.45 (d, J=3.3 Hz, 1/6H), 7.18-7.09 (m, 3H), 7.02-6.98 (m, 1H), 4.77-4.74 (m, 5/6H), 4.71 (d, J=2.4 Hz, 1/6H), 3.63-3.38 (m, 4H, overlapped two singlet at 3.46, 3.44), 2.93-2.89 (m, 1H), 2.35-2.15 (m, 6H, overlapped singlet at 2.32), 2.06-1.99 (m, 1H), 1.26-1.43 (m, 8H, overlapped singlet at 1.20).

### EXAMPLE 4

### Preparation of 3-(2-((2S,4S)-2-cyano-4-fluoropyrrolidin-1-yl)-2-oxoethylamino)-3-methyl-N-((1S,2S)-2-(5-(trifluoromethyl)-1,2,4-oxadiazol-3-yl)cyclopropyl)butanamide

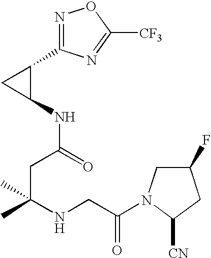

The title compound was prepared in a similar manner as described in Example 3.

¹H NMR (CDCl₃) (3/1 mixture of trans/cis amide isomers): 8.81 (dd, J=11.7, 3.3 Hz, 3/4H), 8.57 (br d, J=11.7 Hz, 1/4H), 5.54 (t, J=3.3 Hz, 3/8H), 5.46 (t, J=3.3 Hz, 1/8H), 5.37 (t, J=3.3 Hz, 3/8H), 5.28 (t, J=3.3 Hz, 1/8H), 4.96 (d, J=9.0 Hz, 3/4H), 4.84 (d, J=9.0 Hz, 1/4H), 3.92 (dd, J=23.4, 23.1 Hz, 3/4H), 3.78 (d, J=3.9 Hz, 1/4H), 3.74 (d, J=3.9 Hz, 1/4H), 3.68-3.62 (m, 3/4H), 3.49-3.28 (m, 3H), 2.79 (t, J=15.6 Hz, 1/4H), 2.71 (t, J=15.6 Hz, 3/4H), 2.34-2.25 (m, 4H, overlapped singlet at 2.28), 1.58-1.46 (m, 2H), 1.17 (s, 3H), 1.16 (s, 3H).

### EXAMPLE 5

### Preparation of N-((1S,2R)-2-(3-chlorophenyl)cyclopropyl)-3-(2-((2S,4S)-2-cyano-4-fluoropyrrolidin-1-yl)-2-oxoethylamino)-3-methylbutanamide

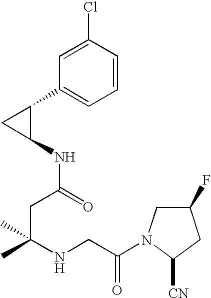

The title compound was prepared in a similar manner as described in Example 3.

¹H NMR (CDCl₃) (4/1 mixture of trans/cis amide isomers): 8.41 (br d, J=3.0 Hz, 4/5H), 8.15 (br s, J=3.0 Hz. 1/5H), 7.17-6.99 (m, 4H), 5.52 (t, J=3.3 Hz, 2/5H), 5.42 (t, J=3.3 Hz, 1/10H), 5.35 (t, J=3.3 Hz, 2/5H), 5.26 (t, J=3.3 Hz, 1/10H), 4.96 (d, J=9.3 Hz, 4/5H), 4.92 (d, J=9.3 Hz, 1/5H), 3.91 (dd, J=23.4, 23.1 Hz, 4/5H), 3.77 (d, J=3.6 Hz, 1/5H), 3.73 (d, J=3.9 Hz, 1/5H), 3.65-3.61 (m, 4/5H), 3.38 (q like, J=16.5 Hz, 2H), 2.94-2.88 (m, 1H), 2.76 (t, J=15.3 Hz, 1/5H), 2.69 (t, J=15.3 Hz, 4/5H), 2.43-2.22 (m, 3H, overlapped singlet at 2.27), 2.05-1.99 (m, 1H), 1.24-1.16 (m, 8H, overlapped singlet at 1.16).

### EXAMPLE 6

### Preparation of 3-(2-((2S,4S)-2-cyano-4-fluoropyrrolidin-1-yl)-2-oxoethylamino)-N-((1S,2R)-2-(4-methoxyphenyl)cyclopropyl)-3-methylbutanamide

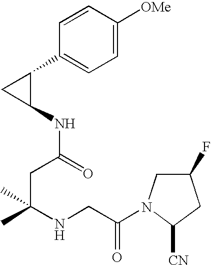

The title compound was prepared in a similar manner as described in Example 3.

¹H NMR (CDCl₃) (3/1 mixture of trans/cis amide isomers): 8.29 (br d, J=3.3 Hz, 3/4H), 8.00 (br s, J=3.3 Hz. 1/4H), 7.08 (d, J=8.4 Hz, 2H), 6.80 (d, J=8.4 Hz, 2H), 5.50 (t, J=3.0 Hz, 3/8H), 5.42 (t, J=3.0 Hz, 1/8H), 5.33 (t, J=3.0 Hz, 3/8H), 5.24 (t, J=3.0 Hz, 1/8H), 4.97 (d, J=8.8 Hz, 1/4H), 4.95 (d, J=8.8 Hz, 3/4H), 3.96-3.54 (m, 5H, overlapped singlet at 3.76), 3.40 (q like, J=16.5 Hz, 2H), 2.88-2.82 (m, 1H), 2.73 (t, J=15.6 Hz, 1/4H), 2.66 (t, J=15.6 Hz, 3/4H), 2.45-2.23 (m, 3H, overlapped singlet at 2.28), 2.0-1.97 (m, 1H), 1.19-1.09 (m, 8H, overlapped singlet at 1.18).

### EXAMPLE 7

### Preparation of 3-(2-((2S,4S)-2-cyano-4-fluoropyrrolidin-1-yl)-2-oxoethylamino)-N-((1S,2R)-2-(3-fluorophenyl)cyclopropyl)-3-methylbutanamide

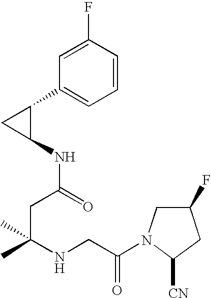

The title compound was prepared in a similar manner as described in Example 3.

¹H NMR (CDCl₃) (3/1 mixture of trans/cis amide isomers): 8.43 (br d, J=3.3 Hz, 3/4H), 8.42 (br s, J=3.3 Hz, 1/4H), 7.20 (q like, J=7.2 Hz, 1H), 6.94-6.81 (m, 3H), 5.51 (t, J=3.3 Hz, 3/8H), 5.43 (t, J=3.3 Hz, 1/8H), 5.34 (t, J=3.3 Hz, 3/8H), 5.26 (t, J=3.3 Hz, 1/8H), 4.95 (d, J=9.3 Hz, 1H), 3.91 (dd, J=23.7, 23.4 Hz, 3/4H), 3.78 (d, J=3.6 Hz, 1/4H), 3.74 (d, J=3.9 Hz, 1/4H), 3.66-3.61 (m, 3/4H), 3.39 (q like, J=16.5 Hz, 2H), 2.95-2.88 (m, 1H), 2.74 (t, J=15.3 Hz, 1/4H), 2.67 (t, J=15.3 Hz, 3/4H), 2.45-2.22 (m, 3H, overlapped singlet at 2.27), 2.10-1.98 (m, 1H), 1.28-1.17 (m, 8H, overlapped singlet at 1.20).

### EXAMPLE 8

### Inhibition of DDP-IV Activities

DPP-IV was purified from human semen according to the method described in de Meester et al. (de Meester et al. (1996) J. Immun. Method 189: 99-105) with minor modifications. Briefly, the semen was diluted with 50 ml of phosphate buffered saline (PBS) and centrifuged at 900 xg for 10 minutes. The supernatant was centrifuged again at 105,000 xg for 120 minutes to separate prostasomes and seminal plasma. The prostasomes, i.e., pellets, and the seminal plasma, i.e., supernatant, were both used for further purification of DPP-IV. The pellets were washed twice with 20 mM Tris-HCl (pH 7.4), and then incubated in 20 mM Tris-HCl (pH 7.4), 1% Triton X-100 for 1 hour at 4° C. The resulting solution was centrifugated at 40,000 xg for 10 minutes to remove prostasomes debris before dialyzed against 20 mM Tris-HCl (pH 7.4), 70 mM NaCl, and 0.1% Triton X-100. The solution was then passed through a DEAE-Sepharose fast flow column (2.6×10 cM) equilibrated with 20 mM Tris-HCl (pH 7.4), 70 mM NaCl and 0.1% Triton X-100 at a flow rate of 2 ml/min. The column was subsequently eluted with 300 ml NaCl (70 to 350 mM) with a linear gradient at a flow rate of 3 ml/min. Positive fractions were pooled and adjusted to pH 8.0 by 0.5 M Tris-HCl (pH 8.0) before applied to an adenosine deaminase-Sepharose columns. The column was prepared as described in de Meester et al. After the column was washed with 10 column volumes of equilibration buffer and then with an equal amount of 50 mM Tris-HCl (pH 7.4) containing 0.5 M NaCl and 0.1% Triton X-100, DPP-IV was eluted with 2 mM Tris-HCl (pH 8.0) containing 0.1% Triton X-100. The supernatant was denatured in 20 mM Tris-HCl (pH 7.4), 1% Tris X-100 for 1 hour at 4° C. The resulting solution was handled as described above to obtain purified DPP-IV.

The kinetic constant of DPP-IV was measured as follows:

All reactions were carried out in PBS using H-Gly-Pro-pNA as a substrate in the presence of 10 nM DPP-IV. The reactions were monitored and measured at OD 405 nm. The initial rate was measured when less than 10% substrate was depleted. The steady state parameters, k_{cat}(=Vₘₐₓ/[E]) and Kₘ, were determined from initial velocity measurements at 0.5-5 Kₘ of the substrate concentrations for the first 300 seconds. Lineweaver-Burk plots were obtained using non-linear regression of the classic Michaelis-Menten equation (equation 1) to obtain Kₘ values. The k_{cat} was calculated from Vₘₐₓ/[E] with the molecular weight of DPP-IV taken as 85,000.
V₀=Vₘₐₓ[S]/(Kₘ+[S]) (equation 1)

where V₀ is the initial velocity, [S] is the substrate concentration, Vₘₐₓ is the maximum velocity and Kₘ is the Michaelis constant. Correlation coefficients better than 0.990 were obtained throughout.

A number of compounds of this invention were tested for their IC₅₀ values for inhibiting DPP-IV. The tested were carried out at 37° C. in 20 mM Tris-HCl (pH 8.0) or in PBS, with purified human semen DPP-IV. The substrate used in the tested was 500 uM H-Gly-Pro-pNA. For each compound, different concentrations were assayed to generate data points, from which the IC₅₀ value was calculated using the Sigma plot. All tested compounds exerted inhibitory activities against DPP-IV. Surprisingly, some of the tested compounds had the IC₅₀ values lower than 10 nM.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to pyrrolidine compounds of this invention also can be made, screened for their inhibitory activities against DPP-IV and treating Type II diabetes and used to practice this invention. Thus, other embodiments are also within the claims.

What is claimed is:

1. A compound of the following formula: 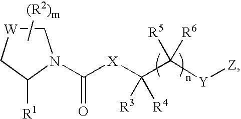
wherein
R¹ is H or CN;
R² is H, halo, nitro, cyano, amino, hydroxy, alkyl, haloalkyl, alkoxy, aryloxy, aralkyl, cyclyl, heterocyclyl, aryl, or heteroaryl;
each of R³, R⁴, R⁵, and R⁶, independently, is H, halo, nitro, cyano, amino, hydroxy, alkyl, haloalkyl, alkoxy, aryloxy, aralkyl, cyclyl, heterocyclyl, aryl, or heteroaryl; or R³ and R⁴, together with the carbon atom to which they are attached, or R⁵ and R^{6,} together with the carbon atom to which they are attached, are a 3-8 membered ring, optionally having 1 or 2 heteroatoms and optionally substituted with halo, CN, NO₂, —OR^{a}, alkyl, aryl, heteroaryl, haloalkyl, —OR^{a}, —C(O)R^{a}, —SR^{a}, —S(O)R^{a}, —S(O)₂R^{a}, —NR^{a}R^{a′}, —C(O)OR^{a}, —C(O)NR^{a}R^{a′}, —OC(O)R^{a}, —NR^{a}C(O)R^{a′}, —NR^{a}C(O)OR^{a′}, or —NR^{a}C(O)NR^{a′}R^{a″}, or optionally fused with one of cyclyl, heterocyclyl, aryl, and heteroaryl, each of R^{a}, R^{a′}, and R^{a″}, independently, being H, alkyl, or aryl;
m is 0, 1, 2, 3, 4, or 5;
n is 0, 1, or 2;
W is CR^{b}R^{b′}, NR^{b}, O, or S, in which each of R^{b} and R^{b′}, independently, is H, halogen, alkyl, or aryl;
X is O, S, or CR^{c}(NR^{c′}R^{c″}), in which each of R^{c}, R^{c′}, and R^{c″}, independently, is H, alkyl, or aryl;
Y is 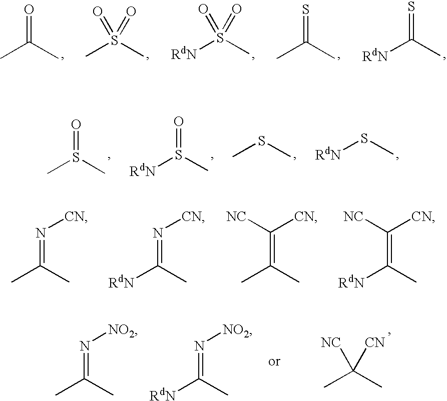
in which R^{d}is H, alkyl, or aryl; and
Z is NR^{e}R^{e′}, in which each of R^{e} and R^{e′}, independently, is H, alkyl, alkoxyalkyl, haloalkyl, cyclyl, heterocyclyl, aryl, aralkyl, heteroaryl, or heteroaralkyl; or NR^{e}R^{e′}, together, is a 3-8 membered ring having 1 or 2 heteroatoms, optionally substituted with halo, CN, NO₂, —OR′, alkyl, aryl, heteroaryl, haloalkyl, —OR, —C(O)R′, —SR′, —S(O)R′, —S(O)₂R′, —NR′R″, —C(O)OR′, —C(O)NR′R″, —OC(O)R′, —NR′C(O)R″, —NR′C(O)OR″, or —NR′C(O)NR″R′″, or optionally fused with one of cyclyl, heterocyclyl, aryl, and heteroaryl, each of R′, R″, and R′″, independently, being H, alkyl, or aryl.

2. The compound of claim 1, wherein each of R³, R⁴, R⁵, and R⁶, independently, is H, halo, nitro, cyano, amino, hydroxy, alkyl, haloalkyl, alkoxy, aryloxy, aralkyl, cyclyl, heterocyclyl, aryl, or heteroaryl.

3. The compound of claim 2, wherein X is CHNH₂.

4. The compound of claim 3, wherein n is 1.

5. The compound of claim 4, wherein each of R³ and R⁴ is CH₃ and each of R⁵ and R⁶ is H.

6. The compound of claim 4, wherein each of R³, R⁴, R⁵, and R⁶ is H.

7. The compound of claim 4, wherein R³ is CH₃ and each of R⁴, R⁵, and R⁶ is H.

8. The compound of claim 3, wherein n is 0.

9. The compound of claim 8, wherein each of R³ and R⁴ is CH₃ and each of R⁵ and R⁶ is H.

10. The compound of claim 8, wherein R is CH₃ and each of R⁴, R⁵, and R⁶ is H.

11. The compound of claim 1, wherein R³ and R⁴ together with the carbon atom to which they attached are a cyclopropyl ring.

12. The compound of claim 11, wherein X is CHNH₂.

13. The compound of claim 12, wherein n is 1.

14. The compound of claim 1, wherein n is 1.

15. The compound of claim 1, wherein the compound is

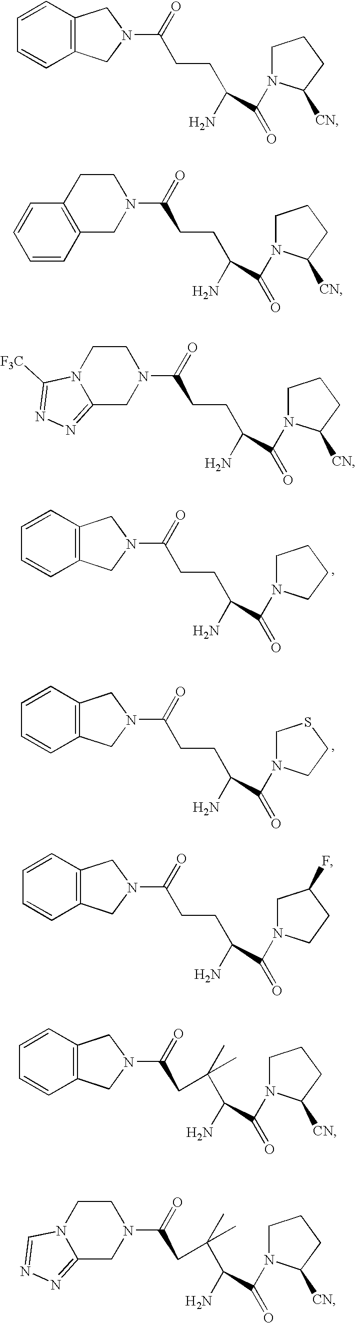

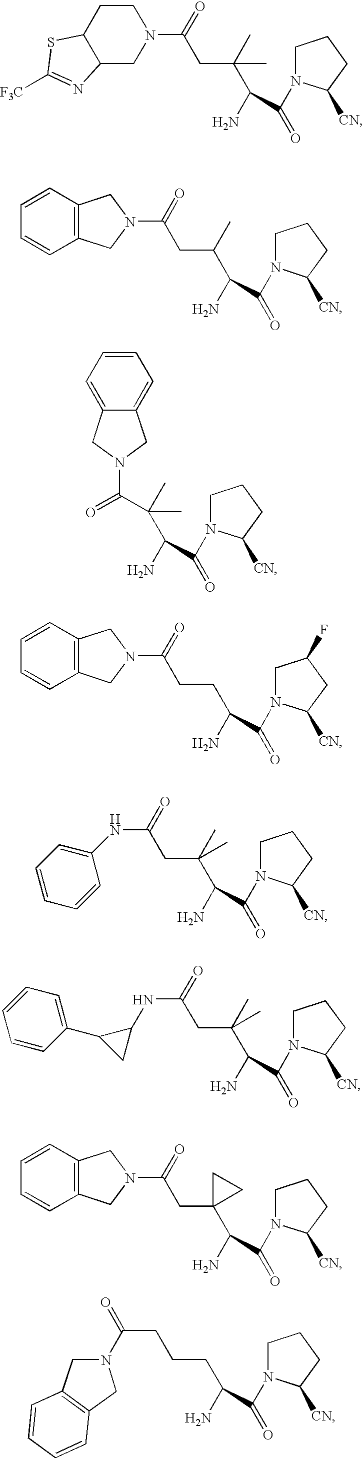

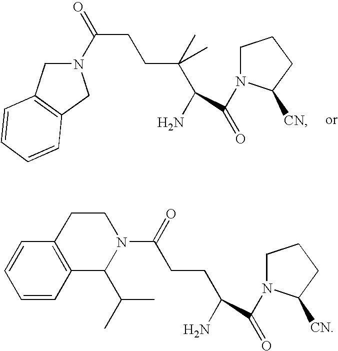

16. A compound of the following formula: 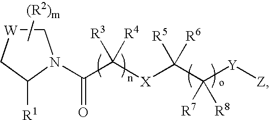
wherein
R¹ is H or CN;
each of R², R³, R⁴, R⁵, R⁶, R⁷, and R⁸, independently, is H, halo, nitro, cyano, amino, hydroxy, alkyl, haloalkyl, alkoxy, aryloxy, aralkyl, cyclyl, heterocyclyl, aryl, or heteroaryl;
m is 0, 1, 2, 3, 4, or 5;
n is 0, 1, 2, 3, or 4;
o is 0, 1, 2, or 3;
W is CR^{a}R^{a′}, NR^{a}, O, or S, in which each of R^{a} and R^{a}, independently, is H, halogen, alkyl, or aryl;
X is NR^{b}, in which R^{b} is H, alkyl, or aryl;
Y is 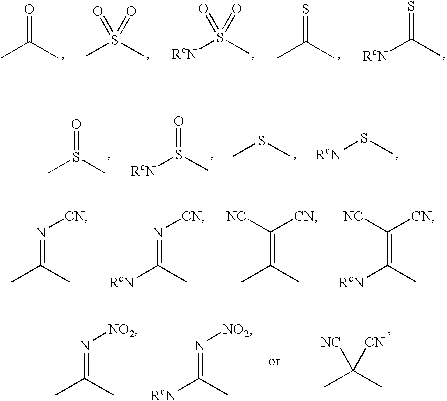
in which R^{c} is H, alkyl, or aryl; and
Z is NR^{d}R^{d′}, in which R^{d} is a 3-8 membered monocyclic ring optionally substituted with halo, CN, NO₂, —OR′, alkyl, aryl, heteroaryl, haloalkyl, —OR′, —C(O)R′, —SR′, —S(O)R′, —S(O)₂R′, —NR′R″, —C(O)OR′, —C(O)NR′R″, —OC(O)R′, —NR′C(O)R″, —NR′C(O)OR″, or —R′C(O)NR″R′″; and R^{d′} is H, alkyl, alkoxyalkyl, haloalkyl, aralkyl, or heteroaralkyl; each of R′, R″, and R′″, independently, being H, alkyl, or aryl.

17. The compound of claim 16, wherein R^{d} is a cyclopropyl ring.

18. The compound of claim 17, wherein the cyclopropyl ring is substituted with an aryl or heteroaryl group.

19. The compound of claim 16, wherein n is 1 and o is 1.

20. The compound of claim 19, wherein R^{d} is a cyclopropyl ring.

21. The compound of claim 20, wherein the cyclopropyl ring is substituted with an aryl or heteroaryl group.

22. The compound of claim 21, wherein each of R³, R⁴, R⁷, and R⁸ is H and each of R⁵ and R⁶ is CH₃.

23. The compound of claim 22, wherein X is NH.

24. The compound of claim 23, wherein W is CH₂.

25. The compound of claim 23, wherein W is CHF.

26. The compound of claims 21, where in X is NH.

27. The compound of claim 16, wherein n is 1, o is 1, each of R³, R⁴, R⁷, and R⁸ is H, and each of R⁵ and R⁶ is CH₃.

28. The compound of claim 16, wherein the compound is

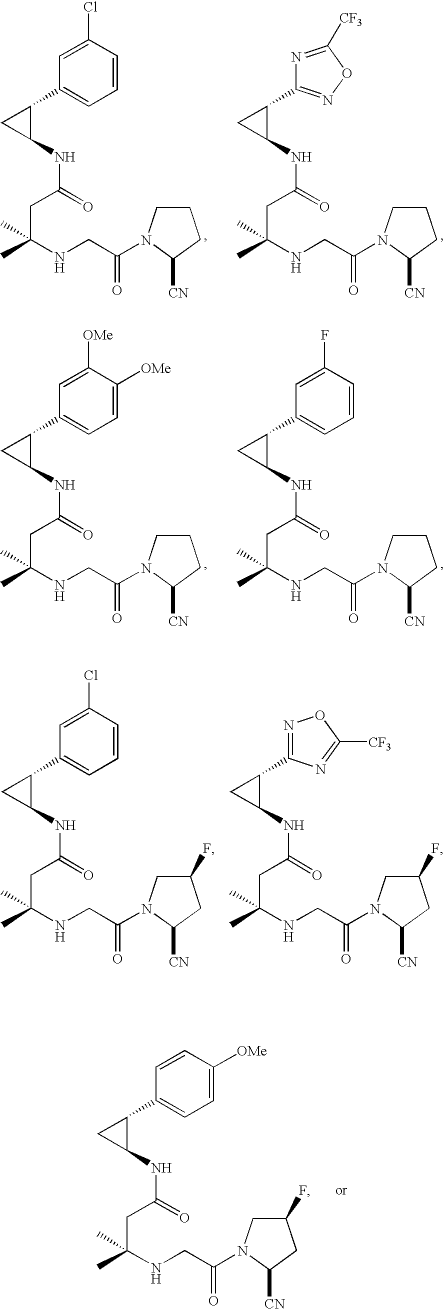

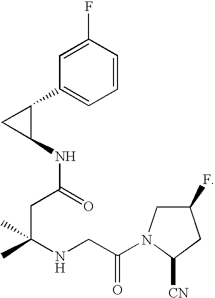

29. A method of inhibiting dipeptidyl peptidase IV, comprising contacting dipeptidyl peptidase IV with an effective amount of a compound of claim 1.

30. A method of inhibiting dipeptidyl peptidase IV, comprising contacting dipeptidyl peptidase IV with an effective amount of a compound of claim 16.

31. A method of treating Type II diabetes, comprising administering to a subject in need thereof an effective amount of a compound of claim 1.

32. A method of treating Type II diabetes, comprising administering to a subject in need thereof an effective amount of a compound of claim 16.
